(19) United States
(12) Patent Application Publication
(10) Pub. No.: US 2008/0254054 A1
(43) Pub. Date: Oct. 16, 2008

(54) Fungal Material Stabilisation

(75) Inventors: Trevor Antony Jackson, Christchurch (NZ); Jayanthis Swaminathan, Christchurch (NZ); Travis Robert Glare, Christchurch (NZ); Tracey Lee Nelson, Christchurch (NZ)

Correspondence Address: KNOBBE MARTENS OLSON & BEAR LLP, 2040 MAIN STREET, FOURTEENTH FLOOR, IRVINE, CA, 92614, US

(73) Assignee: Encoate Holdings Limited, Hamilton (NZ)

(21) Appl. No.: 11/914,119

(22) PCT Filed: May 11, 2006
(86) PCT No.: PCT/NZ06/00107

§ 371 Date: Jun. 5, 2008

(30) Foreign Application Priority Data
May 11, 2005 (NZ) 539962

(51) Int. Cl. A61K 36/00; C12N 1/14; A01N 63/04

(52) U.S. Cl. 424/195.15; 435/254.1; 435/260; 504/117

(57) ABSTRACT

A composition and methods of production are described including fungal material, a solid substrate and a biopolymer composition. The composition is described as being stable when stored for at least 7 months and also reduces dust losses normally attributed to fungal compositions. Uses are described for the composition including pest and weed treatments.

### TECHNICAL FIELD

The invention relates to methods of stabilising fungal material. More specifically, the invention relates to methods of producing stabilised forms of fungal spores, mycelia, and/or sporophores by use of a biopolymer composition.

### BACKGROUND ART

Fungal material such as spores, sporophores and mycelia are presently used in biopesticide and mycoherbicide applications for the control of pests and weeds. Fungi treatment agents have value to manufacturers and users as they provide an environmentally friendly alternative to chemical treatments.

Typical fungi used in biocontrol agent compositions include fungi from the classes: Metarhizium, Beauveria, Sclerotinia, Paecilomyces, Trichoderma, and Fusarium to name a few.

The primary existing method of production of fungi material for use in such applications is to:
(a) grow the fungi on a solid substrate, for example rice grains which provide the growth nutrient;
(b) once the fungus has grown, the rice and spores are separated by placing the mixture on a shaker and the fungal spores are shaken off the grains and collected, or washed off.
(c) spores are then held in a sealed bag or container to prevent contamination or growth of mycelia.

In one alternative, the fungus is not allowed to sporulate and the mycelia is collected. This may include the sporophore stage, which is formed presporulation.

Assuming ideal conditions for the above process and mixing, fungi may continue to be viable from this method for time periods of up to 6 months, depending on the fungus and storage conditions.

However, there are problems with the above method broadly split between issues surrounding viability of the fungi over time and issues surrounding labour and handling.

Viability issues include the fact that:
In some cases, existing methods require that the fungal product be refrigerated to remain viable.
Dried spores are very light and easily disturbed by air movement.
As the fungal product is hydroscopic, the product must be carefully sealed and, when not in a sealed container, handled carefully to avoid moisture exposure.
During separation from solid substrate, spores need to be handled very gently as excessive shaking of the grains reduces fungal material viability.

It should be appreciated that if the viability is reduced, the commercial usefulness of the spores in products such as biopesticides may be dramatically reduced.

Labour and handling issues include the fact that:
Careful handling during the separation step is necessary.
The product does not flow well as it is a fine powdery product that is at least partially wet.
Sterile handling conditions are required throughout the process.
During separation of solid substrate from fungi, it is relatively difficult to avoid ‘contamination’ of the collected fungal spores by portions of solid substrate.
The process must be carried out in an environment with a negative air pressure to prevent contamination of the spores with other fungi or bacteria.
Given the powdery nature of the product, dust from the fungal product may be inadvertently inhaled by the person completing the separation process.

A further problem with the existing method is that it is only marginally profitable for products manufactured on a commercial scale. As an illustration, a Beauveria biopesticide for use in pastoral agriculture is not commercially viable when the product price rises above $40/hectare however, the cost of producing the biopesticide is $30/hectare before any other costs are applied leaving little profit margin.

There are numerous references in the literature to methodologies for fungal production (see references listed at the back of this specification) and a number of patents also outline methods of production (for example, U.S. Pat. No. 4,512,103, U.S. Pat. No. 4,530,834 and U.S. Pat. No. 6,143,549). However, the focus of such publications tends to be around managing inputs such as nutrients to increase spore growth, rather than manipulating the final stages of production to capture spores and preserve them in a more efficient manner. In all cases, managing contamination remains a major issue.

It is therefore an object of the present invention to address the foregoing problems or at least to provide the public with a useful choice.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

Further aspects and advantages of the present invention will become apparent from the ensuing description which is given by way of example only.

### DISCLOSURE OF INVENTION

For the purposes of this specification the words ‘stable’, ‘stability’, ‘viable’ and ‘viability’ will be used interchangeably and refer to the maintenance of spore viability and cellular integrity under conditions (temperature, pH, enzyme reactions etc.) under which spore viability would normally deteriorate.

According to one aspect of the present invention there is provided a composition including:
(a) fungal material selected from: spores, sporophores, mycelia and combinations thereof;
(b) at least one solid substrate; and,
(c) a biopolymer composition.

According to a further aspect of the present invention there is provided a method of producing a stable fungal composition including the steps of:
(a) mixing at least one solid substrate with an inoculum of fungal material selected from: spores, sporophores, mycelia, and combinations thereof;
(b) allowing the fungal inoculum to grow; and,
(c) separating the fungal material grown during step (b) from the solid substrate;
(d) mixing the collected fungal material from step (c) with a porous solid substrate;
(e) at least partially encapsulating the fungal material and porous solid substrate within a biopolymer composition.

According to a further aspect of the present invention there is provided a method of producing a stable fungal composition including the steps of:
(a) mixing at least one solid substrate with an inoculum of fungal material selected from: spores, sporophores, mycelia, and combinations thereof;
(b) allowing the fungal inoculum to grow more fungal material; and,
(c) at least partially encapsulating the solid substrate and grown fungal material from step (b) within a biopolymer composition.

According to a further aspect of the present invention there is provided the use of a composition including:
(a) fungal material selected from: spores, sporophores, mycelia and combinations thereof;
(b) at least one solid substrate; and,
(c) a biopolymer composition;
   for control of insect pests.

According to a further aspect of the present invention there is provided the use of a composition including:
(a) fungal material selected from: spores, sporophores, mycelia and combinations thereof;
(b) at least one solid substrate; and,
(c) a biopolymer composition;
   for control of weeds.

The above compositions, methods and uses have been found by the inventors to result in compositions that are shelf stable at standard atmospheric conditions such as at room temperature (20° C.) for many months. This time period for stability is considerably higher than that found for most compositions which normally deteriorate or lose viability under such conditions. The methods described also have a number of other advantages which should become apparent to those skilled in the art, one of which is the fact that there is no need to separate solid substrate from the fungi and reduced dust losses as the composition of the present invention prevents dust formation.

For the purposes of further description, reference will be made to fungal spores. This should not be seen as limiting as it should be appreciated that other reproductive cellular material may also be collected and maintained viable by the present invention including, but not limited to, conidia, mycelia, sporophores and the like.

In the inventor's experience, the viability of the stabilised fungal material remains consistent when stored at 20° C. for at least 7 months.

The inventors have also noted a considerable advantage form the present invention being reduced amounts of dust being formed. Besides the health and handling issues that reduced dust formation addresses, losses in fungal material from the composition are also reduced. This is because the dust formed is primarily fungal conidia. By lowering these losses, the composition when used will have a greater efficacy than would be the case of the conidia had been removed as dust. More specifically, the rate of conidial loss is reduced from approximately 40% to approximately 5% compared to traditional methods where no biopolymer composition is used to encapsulate the fungal material and substrate.

Preferably, the fungi may be selected from fungi of the division of hyphomycetes characterised by their production of naked spores or conidia. However, it should be appreciated by those skilled in the art that the invention may be applied to any fungi that reproduces asexually.

More preferably, the fungi genus are selected from the group consisting of: Beauveria; Phytophthora; Celletotrichum; Metarhizum; Sclerotinia; Paecilomyces; Trichoderma; Fusarium; and combinations thereof. Most preferably, the fungi may be of the species of Beauveria bassiana. Genus and species described are provided by way of example only and other genera that may have useful properties and require stabilisation may also be encompassed within the invention as described. Specific embodiments envisaged by the inventors include selection of fungi that control weed and pest growth for use in agricultural applications.

Preferably, the pests controlled by the fungi include: soil-dwelling scarabs, beetles and weevil adults and larvae; caterpillars, cicadas, wasps, ants and termites.

Preferably, the weeds controlled by the fungi include: herbaceous pasture weeds; herbaceous crop weeds; herbaceous weeds of fine turf and amenity areas; woody weeds of pastures and natural areas; wilding trees.

Preferably, the herbaceous pasture weeds are giant buttercup, Californian thistle and ragwort.

Preferably, the herbaceous crop weeds are nightshades in pea crops.

Preferably, the herbaceous weeds of fine turf and amenity areas are dandelion, cat's ear, hawkbit, and hawksbeard.

Preferably, the woody weeds of pastures and natural areas are gorse and broom.

Preferably, the wilding trees are willows and poplars.

Preferably, the biopolymer composition includes: water and at least one gum. More preferably the biopolymer composition also includes a surfactant.

Preferably, the water is distilled and substantially sterile.

Preferably, the gum is a polysaccharide gum. More preferably, the gum is selected from the group consisting of: xanthan gum, acacia gum, guar gum, gellan gum, locust bean gum and combinations thereof.

Preferably, surfactants are selected form the group consisting of: t-Octylphenoxypolyethoxyethanol (Triton X-100™); Polyoxyethylenesorbitan (Tween 80™), and combinations thereof. In one embodiment, the surfactant is in dilute concentrations ranging from 0.01% wt to 0.1% wt. More preferably the concentration is approximately 0.05% wt. It should be appreciated that the amount of surfactant used may vary dependent on the fungal material and other aspects such as the solid substrate chosen or even distilled water used.

Preferably, the solid substrate includes any substantially solid material that may be formed into grains or granules and that provides the fungal inoculum with growth nutrients. More preferably, solid substrates may be selected from the group consisting of: rice grains, cereal grains, starch compounds, sands, gravels, zeolite, pumice, and combinations thereof.

Preferably, where rice grains are used as the solid substrate, they may either be in dried or in wet states. Further, rice grains may be either whole, broken, crushed or a combination of whole, broken and/or crushed states.

Preferably, where cereal grains are used as the solid substrate, they may be selected from the group of grain types consisting of: wheat, barley, millet, maize, and combinations thereof.

Preferably, where starch grains are used as the solid substrate, the starch is tapioca starch.

Preferably, during step (b) of the method, the inoculum and substrate mixture are enclosed within a sealed environment. Preferably, the sealed environment is a plastic bag.

Preferably, step (b) is complete when the desired levels of spores, sporophores, and/or mycelia have been reached. In the inventors' experience, this time period is approximately 1 to 4 weeks although, it should be appreciated that this time period may vary depending on various factors including the type of fungi, solid substrate used and environmental conditions such as temperature.

In one embodiment, the solid substrate and grown fungi from step (b) is dried before step (c) is completed. Drying is preferably completed in air for a time period of approximately 2 to 24 hours at a temperature of approximately 25° C.

Preferably, during step (c) of the method, the solid substrate and grown fungal material is fully encapsulated by coating the biopolymer composition over the substrate and fungal material. Preferably coating is completed by gentle mixing although it is envisaged that no particularly special handling will be required unlike existing methods which require very gentle handling to minimise dust release and ensure spore viability.

It should be appreciated from the above description that there is provided methods to produce compositions that maintain fungal reproductive material such as spores in a viable state for extended periods of time when stored in conditions that would normally be associated with rapid deterioration.

The compositions produced also have the advantage of superior flow and reduced dust formation over existing formulations. This is particularly beneficial for ease of handling and to avoid safety issues surrounding dust inhalation by people handling the fungal product. This also assists to ensure that the composition when used has maximum viability and efficacy.

A further advantage of the above methods is that processing steps may be avoided therefore reducing labour and processing complexity and cost. It should be appreciated that the reduced cost processes described above are advantageous to produce a more commercially viable product.

### BRIEF DESCRIPTION OF DRAWINGS

Further aspects of the present invention will become apparent from the following description which is given by way of example only and with reference to the accompanying drawings in which:

FIG. 1 is a photograph of zeolite granules on which fungi and biopolymer composition have been coated;

FIG. 2 is a graph showing spore viability over time for zeolite granules;

FIG. 3 is a photograph of dried rice grains with fungi encapsulated by biopolymer composition;

FIG. 4 is a graph showing spore viability over time for dried rice grains;

FIG. 5 is a graph showing spore viability over time for dried rice grains compared to undried rice grains;

FIG. 6 is a photograph of broken dried rice grains with fungi encapsulated with biopolymer composition;

FIG. 7 is a graph showing spore viability over time for broken dried rice grains with fungi encapsulated by biopolymer composition;

FIG. 8 is a microscope image of spores from granule formulation shown under fluorescence microscopy after staining with STYO/PI stain;

FIG. 9 is a microscope image of spores from rice shown under fluorescence microscopy after staining with STYO/PI stain;

FIG. 10 is a microscope image of spores from rice coated with biopolymer shown under fluorescence microscopy after staining with STYO/PI stain;

FIG. 11 is a graph showing the long term stability of biopolymer coated rice with (square and diamond points) and of dried rice without a biopolymer coating (triangle points); and,

FIG. 12 shows two photographs demonstrating conidia lost by handling from formulated (12A) and non-formulated (12B) rice.

### BEST MODES FOR CARRYING OUT THE INVENTION

The invention is now described with reference to a series of experiments completed to determine the effectiveness of various methods at producing and maintaining viable fungal material.

### Background

Two different types of formulation were prepared, either as a spore coated granule (Trial 1) or as encapsulation of the spores (Trial 2). Drying requirements and broken versus whole substrates were also tested (Trials 3 and 4) as well as spore viability (Trial 5). Additional trials (Trials 6 to 11) were included to:
Show that the method works for other strains of fungi;
Show that other types of polysaccharide may also be used;
Further data on the stability of the product;
Incorporation onto other types of solid substrate;
Results where the product is used for weed and pest control;
Measures to determine the degree of loss in dust with and without treatment.

The formulations of Trials 1 to 4 were stored in gas transferable bags (GT bags) and shelf life was monitored at 20° C. under standard atmospheric conditions (i.e. atmospheric pressure, non-extreme humidity etc).

A fluorescence stain test was also completed to verify cell viability (Trial 5).

### Trial 1

Zeolite is a porous clay material with absorbent properties. In this trial the potential for using zeolite as a carrier for Beauveria spores is investigated to assist with stabilisation and application of the fungal material in the field.

### Methods

Sterile zeolite granules (approximately 2-4 mm diameter) were coated with Beauveria bassiana spores, as described below.

The formulation was prepared by:
(a) mixing 158 ml of distilled water with 4 grams of xanthan gum to form a biopolymer gel;
(b) 20 grams of harvested B. bassiana spores (1×10¹²/cfu/g were homogenised with 163 ml of sterile distilled water containing 0.05% Triton X-100™ surfactant using a Polytron;
(c) The biopolymer gel and the homogenised spore mixture were then combined to form a spore-gel;
(d) The spore-gel was then added to sterile zeolite granules (845 g) in a mixing bowl whilst the mixer was running mixed to form the end formulation.

The formulation was then stored at 20° C. and viability tested by enumerating samples at monthly intervals using plate counting tests to determine the number of viable cells.

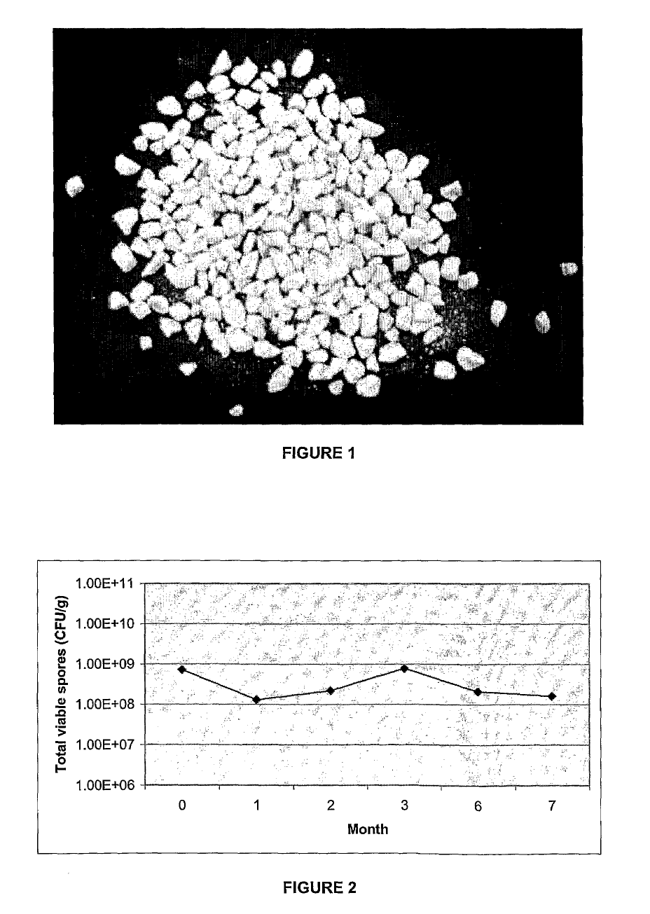

A photograph of the zeolite granule end product is shown in FIG. 1.

### Results

The number of viable colony forming units or cfus (should be equivalent to spores theoretically) immediately after preparation of the formulation was 7.2×10⁸ cfus per gram of granules. This comprised approximately 80% of the theoretical number of spores applied, indicating little loss of viability during processing. The number of viable cfus on the granules continued to remain stable over the following 7-month trial period tested as shown in FIG. 2.

It is the inventors understanding that the variation in spore count noted between sampling times suggests heterogeneity variation between the samples rather than changes in viable spore count.

Further observations noted were that coating Beauveria spores onto zeolite granules results in a product that flows well and is easy to handle with little dust formation. However, a disadvantage noted with this method is that the processing costs of spore extraction are higher than that desired.

### Trial 2

A direct encapsulation method was tested to determine if it is possible to remove the need for harvesting or separation of spores from rice grains.

### Method

Rice grains with spores grown using existing methods were dried and encapsulated using a biopolymer gel.

The biopolymer gel was prepared by mixing 2 grams of xanthan gum (Grindsted@Xanthan Easy Rhodiogel Easy™) in 48 ml of sterile distilled water to give 4% gel strength.

The biopolymer gel was then added to the dried rice grains at a rate of 2% (20 grams gel per kg of rice grains) in the bowl of a mixer operating at a low speed. To ensure efficient mixing, the grains were intermittently melded together with a clean plastic spatula for uniform distribution of the gel. The grains coated with the gel and spores were then air-dried on a clean plastic tray at approximately 25° C. for 2 hours.

### Results

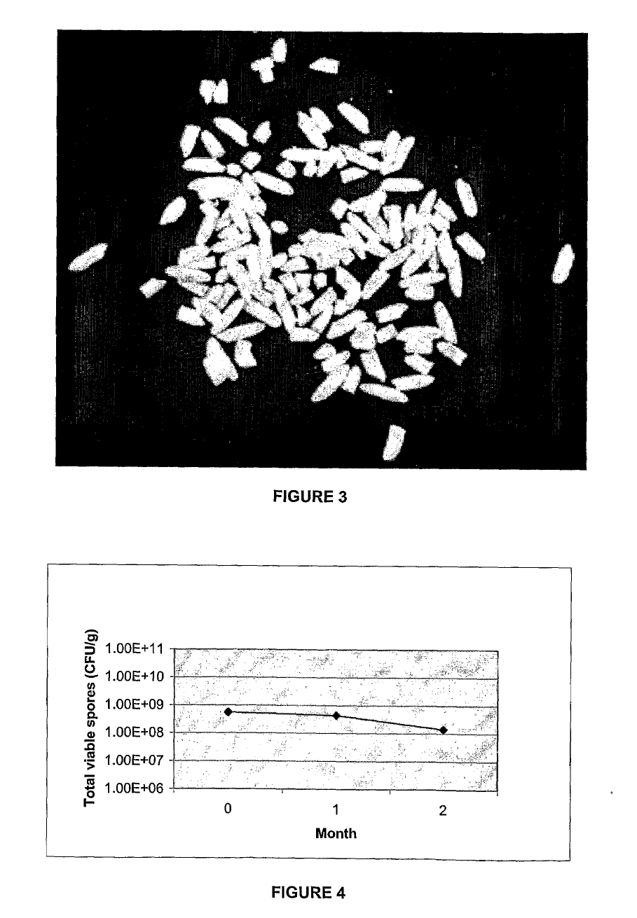

The product obtained is shown in FIG. 3. Good flow characteristics were noted with little dust formed.

The initial count of viable cfus on rice was measured at 8.0×10⁸ cfu/g. Shelf life of the formulation was then tested by measuring the plate count of samples stored at 20° C. taken at monthly intervals. The results on the shelf data obtained (shown in FIG. 4) indicate minimal change in cfus count over the 2 months measured.

An advantage of the above method is that, by incorporating the biopolymer directly onto the grain coating, this method avoids the potential handling problems and associated costs (labour and reduced spore count) of a spore separation step.

### Trial 3

Biopolymer at 4 to 10% gel strength was encapsulated onto rice at a rate of 2 to 10% that was either undried or dried after spore production. These options were tested to investigate the possibility of eliminating the drying step after spore production.

### Method

For both rice samples (dried and undried), 5 grams of xanthan gum (Rhodigel Easy™) was mixed with 45 ml of sterile distilled water containing approximately 0.05% wt Tween 80™ surfactant. The rice grains, both dried (24 hours drying on a clean plastic tray at 25° C.) and undried (taken directly from the production bags), were coated at a rate of 2% gel/grain. The formulations were packed in gas transferable bags and the shelf life was monitored at monthly intervals at 20° C. by measuring plate counts.

### Results

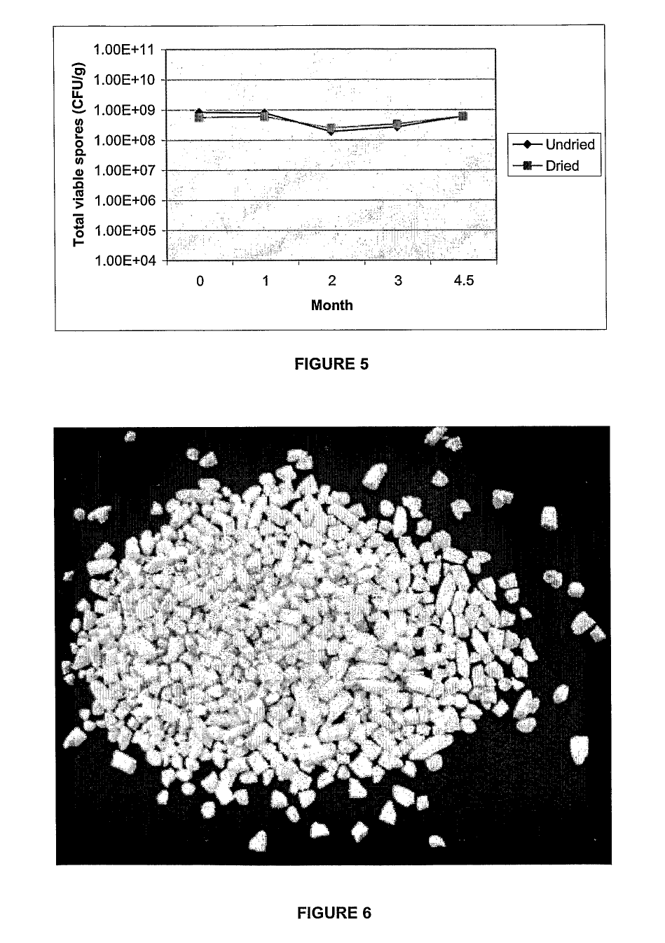

The initial viable number of colony forming units (cfus) for undried and dried rice samples were 8.4×10⁸ cfu/g and 5.6×10⁸ cfu/g, respectively. The results (shown in FIG. 5) indicate that there was little loss in viability as measured by cfu count over the period of 4 months.

The results indicate that it does not matter whether rice was dried or undried at the end of the production process. It is the inventors understanding that the biopolymer in effect dehydrates the grain spore mix. Using biopolymer on undried rice is advantageous as this eliminates a further processing step, saving energy and labour costs as well as simplifying the process.

### Trial 4

Spore production was investigated on broken rice grains to determine if grain shape had an effect on viability.

### Method

The method of preparation was the same as that of Trial 2 (see above) except that broken rice grains were used rather than whole grains as described in Trial 2.

### Results

The end product is shown in FIG. 6. Like other products, good handling characteristics were found including reduced dust and good flow characteristics.

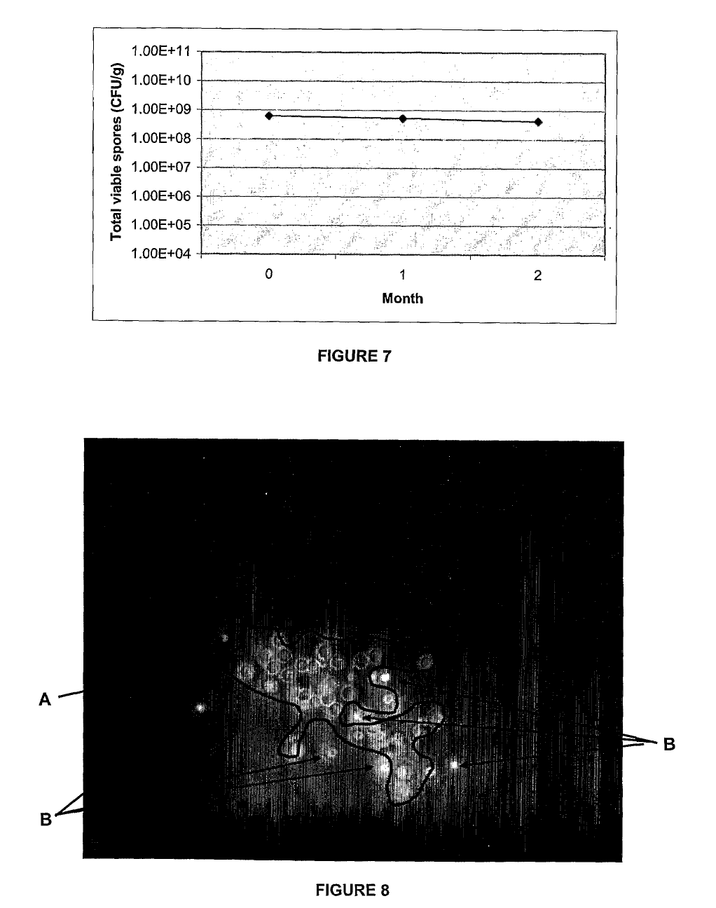

The initial count of viable cfus on the broken rice product was 6.3×10⁸ cfu/g. Shelf life of the formulation stored at 20° C. was tested at monthly intervals and is shown in FIG. 7. After 2 months, shelf life remained above 10⁸ cfu/g showing no effect on viability due to use of broken grains.

The broken rice production and formulation system appears to have a number of advantages over the previously described systems. In addition to reducing dust and improving survival, greater homogeneity is achieved on the broken rice grains as indicated by the consistent viability results. Homogeneity is desirable to deliver the required number of spores to the soil.

### Trial 5

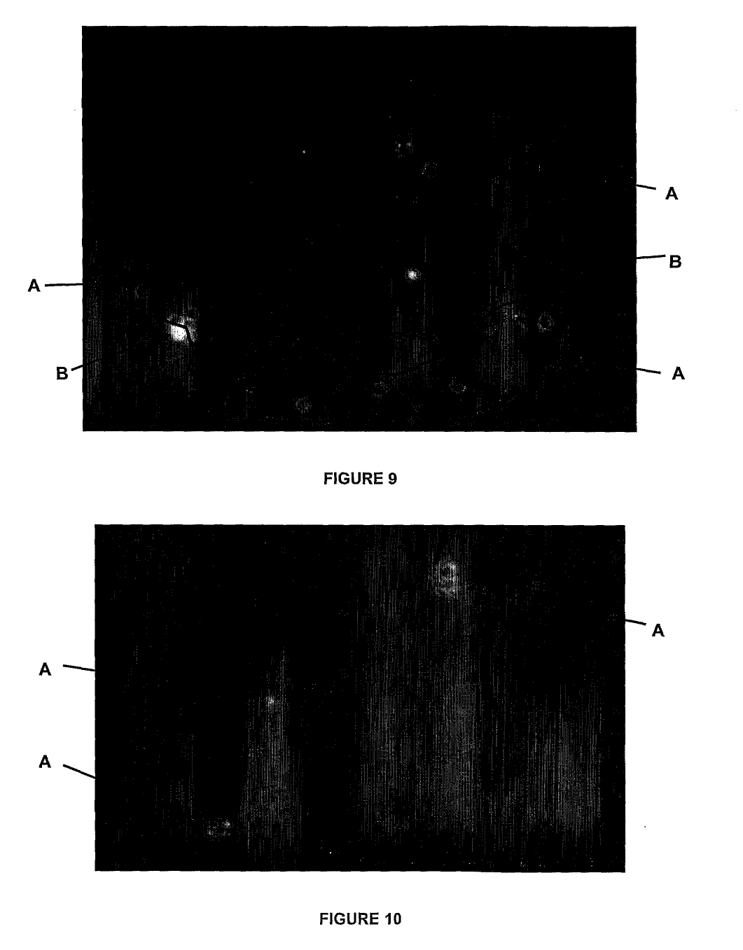

Spore viability in the formulations was confirmed by fluorescence microscopy following staining with STYO/PI dyes (Molecular Probes Ltd). As shown in FIGS. 8 to 10, live cells appear as a green colour (marked A), while dead cells stain red (marked B), due to ingress of propidium iodide. The test confirms that few spores have died in this sample verifying the results found in Trials 1 to 3.

### Trial Results Summary

Shelf life data (cfu/g) for Beauveria bassiana formulations stored at 20° C. were as shown in Table 1 below.

TABLE 1
Shelf life data (cfu/g) for Beauveria bassiana formulations stored at 20° C.

| | | 1 | 2 | 3 | 4.5 | 6 | 7 |
|---|---|---|---|---|---|---|---|
| Formulation | Initial | month | months | months | months | months | months |
| Zeolite - | 7.2 × 10⁸ | 1.3 × 10⁸ | 2.2 × 10⁸ | 7.8 × 10⁸ | | 2.1 × 10⁸ | 1.6 × 10⁸ |
| Trial 1 | | | | | | | |
| Rice - | 5.5 × 10⁸ | 4.4 × 10⁸ | 1.4 × 10⁸ | | | | |
| Trial 2 | | | | | | | |
| Dried Rice - | 8.4 × 10⁸ | 7.8 × 10⁸ | 1.9 × 10⁸ | 2.7 × 10⁸ | 6.1 × 10⁸ | | |
| Trial 3 | | | | | | | |
| Undried | 5.6 × 10⁸ | 4.2 × 10⁷ | 2.5 × 10⁸ | 3.4 × 10⁸ | 6.0 × 10⁸ | | |
| Rice - | | | | | | | |
| Trial 3 | | | | | | | |
| Broken | 6.3 × 10⁸ | 5.3 × 10⁸ | 4.3 × 10⁸ | | | | |
| Rice - | | | | | | | |
| Trial 4 | | | | | | | |

### Trial 6

An additional trial was completed using four strains of fungi grown on rice and coated with biopolymer. The strains tested were, Beauveria caledonica, Metarhizium anisopliae var anisopliae, and Metarhizium anisopliae. Short term shelf life studies were completed at 20° C. to determine that the method works for other fungi strains. The results found were as shown in Table 2:

TABLE 2
Shelf Life Studies for Various Fungal Strains

| | | Viable conidia | |
|---|---|---|---|
| | Initial conidial | after 60 days at | |
| Fungal strain | count (cfu/g) | 20° C. | % survival |
| Metarhizium | 4.3 × 10⁸ | 4.8 × 10⁸ | 100% |
| anisopliae var | | | |
| anisopliae: | | | |
| Biopolymer coated | | | |
| Metarhizium | 4.1 × 10⁶ | 1.2 × 10⁶ | 27% |
| anisopliae: | | | |
| Biopolymer coated | | | |
| Beauveria | 3.5 × 10⁸ | 1.5 × 10⁸ | |
| caledonica | | (90 days) | |
| Formulated | | | |

The above results show that the viability is maintained over time for various strains of fungi and that from these results it should be obvious that other fungi strains would also give similar results. It should also be noted that the inventors found that dust levels from the different strains of fungi were also maintained at lower levels than that expected when no biopolymer treatment is undertaken.

### Trial 7

The above trials were completed using xanthan gum as the biopolymer. It should be appreciated that other polysaccharides may also be used and the following trial is conducted to show use of an alternative polysaccharide.

In this trial the survival of fungal conidia in a gellan biopolymer coating after storage at 20° C. for 7 days was tested. The results are shown below in Table 3.

TABLE 3
Use of Gellan as the Biopolymer Agent

| | Initial conidial | Viable conidia after 7 |
|---|---|---|
| Fungal strain | count (cfu/g) | days at 20° C. |
| Metarhizium anisopliae var | 2.6 × 10⁸ | 1.4 × 10⁸ |
| anisopliae: Biopolymer coated | | |
| Beauveria bassiana: Biopolymer | 6.0 × 10⁸ | 4.7 × 10⁸ |
| coated | | |

The results show that the method may be completed as expected using alternative biopolymers such as gellan.

### Trial 8

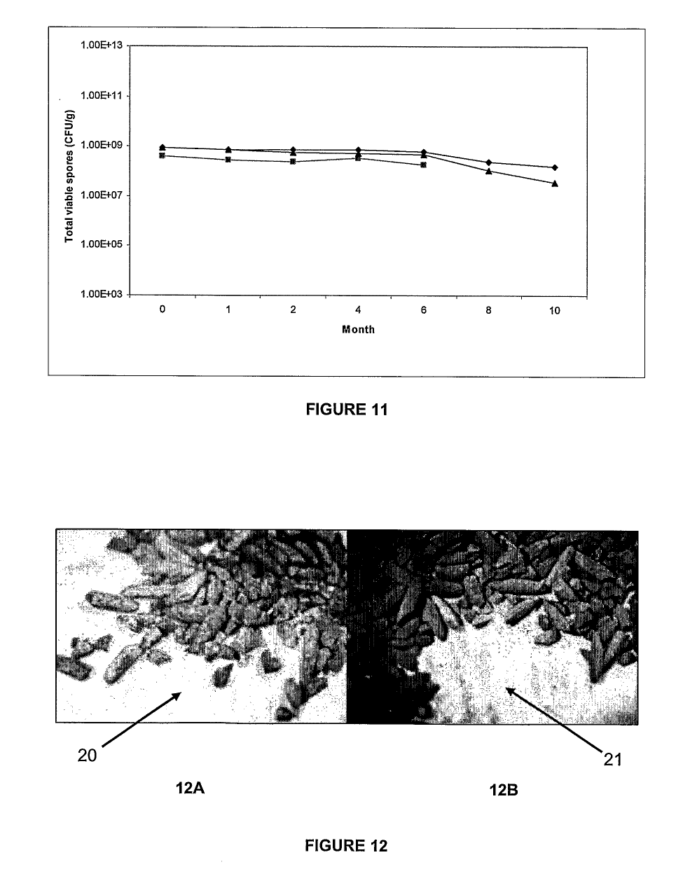

Assessments of the long term stability of biopolymer coated rice with Beauveria bassiana were tested and found to be highly reproducible. Results found were as shown in FIG. 11. Conidial viability remained constant for over seven months. The decline in viability of conidia was greater for rice without the biopolymer coating (triangle points) than for biopolymer coated rice (square and diamond points). This suggests the shelf life of biopolymer coated rice with Beauveria bassiana will be enhanced as expected.

### Trial 9

A variety of additional solid substrates were tested for use with conidial spores.

### Wheat, Barley, Other Grains

Beauveria sp and Metarhizium sp have been successfully grown on wheat and barley both by the applicant.

Formulation with Biopolymer/Zeolite Granules

Spores were extracted from the rice to produce a high density spore powder. The spore powder was incorporated into a biopolymer and successfully coated onto zeolite granules.

### Trial 10

Further trials were completed to verify that the stabilized fungal material does indeed work as expected to control weeds and pests.

Biopolymer stabilised materials were used in field in trials to control the pests Clover Root Weevil and Fullers Rose Weevil.

### Clover Root Weevil (CRW)

Two field trials were established to examine effects of Beauveria bassiana isolate on larval populations of the clover root weevil, Sitona lepidus. One trial compared the biopolymer-coated rice and spores formulation against an emulsion and clay-based granular formulation. Measurements of persistence by soil plating (colony forming units) showed that the new formulation established at reasonable numbers in all trials suggesting stable establishment of Beauveria bassiana at around 10³-10⁴ conidia/g soil, which it should be appreciated is sufficient for larval infection to occur.

Paddock-scale trials were established in a second trial, where Beauveria bassiana was applied as a rice and biopolymer formulation, targeting CRW larvae in the clover root feeding zone. The biopolymer rice formulation was very easy to handle and apply using commercial farm equipment. There was minimal dust when transferring the product from bags to the seed drill, making handling easy and comfortable for the operator. The product was also extremely stable, with no change in spore loading and viability over 6 weeks (trial finished at week 6). From field sampling B. bassiana was recovered from all experimental sites up to 12 weeks post-treatment. Soil loadings appeared to stabilise at around 2×10⁴ CFU/g, and infected cadavers were collected from the treated plots proving effective use.

### Fuller Rose Weevil

The establishment of Beauveria bassiana applied as a granular formulation was assessed in three kiwifruit orchards. Granules were applied at a rate of 70 g/m2.

After three months, B. bassiana was isolated at rates up to 10³ CFU/g soil from treated plots, significantly higher than CFU numbers isolated from untreated plots. The results indicated good potential for the granular formulation of Beauveria bassiana to persist in kiwifruit orchard soils.

### Trial 11

As noted above, a key advantage of the present invention is that the method produces a product that is easy to handle with minimal dust. A trial was completed to show the degree of dust formation.

Biopolymer coated rice prevents dust formation due to displacement of conidia when handling. This is clearly demonstrated in FIG. 12 where the white filter paper is covered with the green conidia FIG. 12B (shown generally by arrow 21) from the unformulated rice compared to FIG. 12A (shown generally by arrow 20) which shows formulated rice.

Formulations of coated and uncoated rice grains with Metarhizium anisopliae conidia were fed into a Duncan seed drill through the seed box and collected into plastic bags. The samples were then weighed and spores washed off the rice in 0.01% Triton-X 100. Haemocytometer counts were used to analyse the number of conidia per gram of rice both before and after the rice was passed through the seed drill. The results found were that the rate of conidial loss is reduced using the invention method from 40% loss to 5% loss. This is understood to be the result of both use of a biopolymer as well as optionally not having to separate fungal material from solid substrate. More examples of the results are shown below in Table 4.

TABLE 4
Conidial Loss Results Due to Dust Formation

| | Conidia lost between |
|---|---|
| | production and after |
| Formulation | drilling |
| M. flavoviride strain IMI - formulated | 13% |
| M. flavoviride strain IMI - non formulated | 36% |
| M. anisopliae strain - formulated | 1% |
| M. anisopliae strain - non formulated | 13% |

It should be appreciated that the above trials show that the present invention provides methods to produce compositions that maintain fungal reproductive material such as spores in a viable state for extended periods of time when stored in conditions that would normally be associated with rapid deterioration. The compositions produced also have the advantage of superior flow and reduced dust characteristics over existing formulations. This is particularly beneficial for ease of handling and to avoid safety problems. A further advantage of the above methods is that processing steps may be avoided therefore reducing labour and processing complexity and cost.

Aspects of the present invention have been described by way of example only and it should be appreciated that modifications and additions may be made thereto without departing from the scope thereof as defined in the appended claims.

### REFERENCES

Burges H D ed. (1998). Formulation of microbial biopesticides: beneficial microorganisms, nematodes and seed treatments. Dordrecht; Netherlands: Kluwer Academic Publishers. 412 pp.
Cherry A J, Jenkins N E, Heviefo G, Bateman R and Lomer C J (1999) Operational and economic analysis of a West African pilot-scale production plant for aerial conidia of Metarhizium spp. for use as a mycoinsecticide against locusts and grasshoppers. Biocon Sci Technol. 9: 35-51.
Dorta B, Bosch A, Arcas J A and Ertola R J (1990). High level of sporulation of Metarhizium anisopliae in a medium containing by-products. Appl Miol Biotechnol 33: 712-715.
Feng M G, Poprawski T J and Khachatourians G G (1994). Production, formulation and application of the entomopathogenic fungus Beauveria bassiana for insect control: current status. Biocon Sci Tech 4: 3-34.
Grimm C (2001). Economic feasibility of a small-scale production plant for entomopathogenic fungi in Nicaragua. Crop Prot 20: 623-630.
Jenkins N E, Heviefo G, Langewald J, Cherry A J and Lomer C J (1998). Development of mass production technology for aerial conidia for use as mycopesticides. Biocon News Inform 19: 21N-31N.
Logan D P, Robertson L N and Milner R J (2000) Review of the development of Metarhizium anisopliae as a microbial insecticide, BioCane™, for the control of greyback canegrub Dermolepida albohirtum (Waterhouse) (Coleoptera: Scarabaeidae) in Queensland sugarcane. Bulletin-OILB-SROP 23:131-137.
McCabe D E and Soper R S (1985). Preparation of an entomopathogenic fungal insect control agent. U.S. Pat. No. 4,530,834. July 23, 3 pp.
Moore D, Douro-Kpindou O K, Jenkins N E and Lomer C J (1996). Effects of moisture content and temperature on storage of Metarhizium flavoviride conidia. Biocontrol Sci Tech 6: 51-61.
Nelson T L, Low A and Glare T R (1996). Large scale production of New Zealand strains of Beauveria and Metarhizium. Proc 49th NZ Plant Prot Conf 257-261.
Pereira R M and Roberts D W (1990) Dry mycelium preparations of entomopathogenic fungi, Metarhizium anisopliae and Beauveria bassiana. J Invertebr Path 56: 39-46.
Reinecke P, Andersch W, Stenzel K and Hartwig J (1990). BIO 1020, a new microbial insecticide for use in horticultural crops. Brighton Crop Prot Conf, Pests and Diseases 1: 49-84.

Samson P R and Milner R J, (1999). Metarhizium-based pesticides for Queensland canegrubs. Proc 7th Aust Conf Grassland Inv Ecol: 92-98.
Shah P A, Aebi M and Tuor U (1998). Method to immobilize the aphid-pathogenic fungus Erynia neoaphidis in an alginate matrix for biocontrol. Appl Environ Microbiol 64: 4260-4263.
Shah P A and Goettell M S eds. (1999) Directory of Microbial Control Products and Services. Microbial Control Division, Soc Invertebr Pathol 31 pp.
Smith S M, Moore D, Karanja L W and Chandi E A (1999). Formulation of vegetable fat pellets with pheromone and Beauveria bassiana to control the larger grain borer, Prostephanus truncatus (Horn). Pesticide Sci 55: 711-718.
Sopp P I, Gillespie A T and Palmer A (1989). Application of Verticillium lecanii for the control of Aphis gossypii by a low-volume electrostatic rotary atomiser and a high-volume hydraulic sprayer. Entomophaga 34: 417-428.

1. A composition including:
(a) fungal material selected from: spores, sporophores, mycelia and combinations thereof; and,
(b) at least one solid substrate;
and characterized in that the mixture is coated in a biopolymer composition.

2. The composition of claim 1 wherein the composition is shelf stable stored at 20° C. for at least 7 months.

3. The composition of claim 1 wherein the rate of conidial loss is reduced from approximately 40% to approximately 5% loss.

4. The composition as claimed in claim 1 wherein the fungal material is selected from fungi of the division of hyphomycetes characterised by their production of naked spores or conidia.

5. The composition as claimed in claim 1 wherein the fungal material is selected from fungi of the genus: Beauveria, Phytophthora, Celletotrichum, Metarhizum, Sclerotinia, Paecilomyces, Trichoderma, Fusarium, and combinations thereof.

6. The composition as claimed in claim 1 wherein the fungal material is from the species Beauveria bassiana.

7. The composition as claimed in claim 1 wherein the biopolymer composition includes water and at least one gum.

8. The composition as claimed in claim 7 wherein the gum is a polysaccharide gum.

9. The composition as claimed in claim 7 wherein the gum is selected from: xanthan gum, acacia gum, guar gum, gellan gum, locust bean gum, and combinations thereof.

10. The composition as claimed in claim 1 wherein the biopolymer composition also includes a surfactant.

11. The composition as claimed in claim 10 wherein the surfactant is selected from: t-Octylphenoxypolyethoxyethanol (Triton X-100™) or Polyoxyethylenesorbitan (Tween 80™) or combinations thereof.

12. The composition as claimed in claim 1 wherein the solid substrate includes any substantially solid material that may be formed into grains or granules and that provides the fungal inoculum with growth nutrients.

13. The composition as claimed in claim 1 wherein the solid substrate is selected from: rice grains, cereal grains, starch compounds, sands, gravels, zeolite, pumice, and combinations thereof.

14. The composition as claimed in claim 1 wherein the solid substrate is rice grains.

15. The composition as claimed in claim 14 wherein the rice grains are whole, broken, crushed or a combination of whole, broken and/or crushed states.

16. The composition as claimed in claim 13 wherein the cereal grains include wheat, barley, millet, maize, and combinations thereof.

17. The composition as claimed in claim 13 wherein the starch is tapioca starch.

18. A composition including fungal material, at least one solid substrate and biopolymer composition that is stable at 20° C. and has reduced conidial loss in the form of dust formation.

19. A method of producing a stable fungal composition including the steps of:
(a) mixing at least one solid substrate with an inoculum of fungal material selected from: spores, sporophores, mycelia, and combinations thereof;
(b) allowing the fungal inoculum to grow; and,
(c) separating and collecting the fungal material grown during step (b) from the solid substrate;
(d) mixing the collected fungal material from step (c) with a porous solid substrate;
(e) at least partially encapsulating the fungal material and porous solid substrate within a biopolymer composition.

20. The method as claimed in claim 19 wherein during step (e) the fungal material and porous solid substrate are both fully encapsulated within the biopolymer composition.

21. The method as claimed in claim 19 wherein porous solid substrate and fungal material from step (d) are dried before step (e) is completed.

22. A method of producing a stable fungal composition including the steps of:
(a) mixing at least one solid substrate with an inoculum of fungal material selected from: spores, sporophores, mycelia, and combinations thereof;
(b) allowing the fungal inoculum to grow more fungal material; and,
(c) at least partially encapsulating the solid substrate and grown fungal material from step (b) within a biopolymer composition.

23. The method as claimed in claim 22 wherein during step (c) the solid substrate and grown fungal material are both fully encapsulated within the biopolymer composition.

24. The method as claimed in claim 22 wherein solid substrate and grown fungal material from step (b) are dried before step (c) is completed.

25. The method as claimed in claim 19 wherein, during step (b) of the method, the inoculum and substrate mixture are enclosed within a sealed environment.

26. The method of claim 25 wherein sealed environment is a plastic bag.

27. The method as claimed in claim 19 wherein step (b) is complete after 1 to 4 weeks.

28. The method as claimed in claim 19 wherein encapsulation is completed by gentle mixing to coat the fungal material and solid substrate with biopolymer composition.

29. (canceled)

30. Use of a composition as claimed in claim 1 for control of insect pests.

31. The use as claimed in claim 30 wherein the insect pests include: soil-dwelling scarabs, beetles and weevil adults and larvae; caterpillars, cicadas, wasps, ants and termites.

32. Use of a composition as claimed in claim 1 for control of weeds.

33. The use of a composition as claimed in claim 32 wherein the weeds include: herbaceous pasture weeds; herbaceous crop weeds; herbaceous weeds of fine turf and amenity areas; woody weeds of pastures and natural areas; wilding trees.

34. Use of a stable fungal composition as claimed in claim 1, substantially as hereinbefore described and with reference to the Trials and Figures.

35. The composition as claimed in claim 1 wherein the composition is stored in at least one gas transferable bag.
